(19) United States
(12) Patent Application Publication
(10) Pub. No.: US 2010/0094179 A1
(43) Pub. Date: Apr. 15, 2010

(54) Optimized Diffraction Zone for Ultrasound Therapy

(75) Inventors: Neill M. Pounder, Germantown, TN (US); Holly V. Ironfield, Lancashire (GB); Javier F. De Ana, Germantown, TN (US); Andrew J. Harrison, Yorkshire (GB)

Correspondence Address: FISH & RICHARDSON P.C.;Smith & Nephew, Inc., 1450 Brooks Road, Memphis, TN, 38116, US

(73) Assignee: Smith & Nephew Inc, Memphis, TN (US)

(21) Appl. No.: 12/520,470

(22) PCT Filed: Dec. 21, 2007
(86) PCT No.: PCT/US07/88748

§ 371 Date: Dec. 22, 2009

Related U.S. Application Data
(60) Provisional application No. 60/871,589, filed on Dec. 22, 2006.

(51) Int. Cl. A61N 7/00

(52) U.S. Cl. 601/2

(57) ABSTRACT

A system and method for ultrasound therapy may include a transducer configured to deliver an ultrasound wave to tissue. Means for manipulating the diffraction pattern alter the transition point between the near field and far field.

### CROSS-REFERENCE TO RELATED APPLICATIONS

This application claims priority to U.S. Provisional Application No. 60/871,589, filed Dec. 22, 2006.

### BACKGROUND

1. Field

The present invention relates generally to ultrasound therapy and more particularly to optimization of ultrasound wave application.

2. Related Art

The output of an ultrasound therapy device is an ultrasound pressure wave that propagates through tissue and into bone to accelerate fracture healing. The ultrasound beam changes with depth due to wave diffraction but the transducer is always positioned in contact with the patient's skin over the fracture site. It has been shown that there is an optimal range of distances at which the transducer should be placed to maximize the biological response. As the transducer needs to be in contact with the skin, an alternative application method needs to be implemented so that the diffraction pattern characteristics that maximize the biological response are located around the fracture site.

This problem has not been previously addressed by the prior art as the optimal range of distances between the fracture site and the transducer have not been investigated.

### SUMMARY OF THE INVENTION

This invention manipulates the diffraction pattern to position the area of maximum biological effect to the ultrasound stimulus where the fracture site is located. This can improve the fracture healing acceleration normally obtained with the ultrasound therapy bone healing device. Furthermore, with some of the embodiments, the ultrasound healing system can be customized to each patient based on the depth of the fracture and the type of fracture for which it is used.

A system for ultrasound therapy may include a transducer configured to deliver an ultrasound wave to tissue. Means for manipulating the diffraction pattern alter the transition point between the near field and far field.

An embodiment may include means for manipulating the diffraction pattern with a block coupled to the transducer. The block may have a speed of sound for propagating waves different from the speed of sound for propagating waves in the tissue.

Alternatively, the speed of sound for propagating waves in the block may greater than the speed of sound of propagating waves in the tissue.

Another embodiment may include means for manipulating the diffraction pattern using a lens.

Alternatively, the lens may be a concave lens.

Another embodiment may include means for manipulating the diffraction pattern using an array of transducers.

In another embodiment, the array of transducers may be concentric.

In yet another embodiment, the array of transducers may be activated by varying the voltage to each element in the array of transducers.

Alternatively, a first element in the array of transducers is activated after a time delay from the activation of a second element in the array of transducers.

Another embodiment includes means for manipulating the diffraction pattern including a first block having a first speed of sound for propagating waves and a second block having a second speed of sound for propagating waves. The second speed of sound for propagating waves is different than the first speed of sound for propagating waves.

In another embodiment, the first block and the second block are concentric.

A method for ultrasound treatment of tissue comprises providing a transducer for propagating ultrasound waves into tissue. The method identifies a depth for treatment. The method manipulates the diffraction pattern of the ultrasound waves such that the transition between the near field and the far field is between the transducer and the identified depth of treatment.

Alternatively, the manipulating step may include the step of propagating the ultrasound signal through a lens.

In another embodiment, the providing step includes providing an array of transducers. The manipulating step includes activating a first element of the array of transducers with a first voltage and a second of the array of transducers with a second voltage. The second voltage is different from the first voltage.

In yet another embodiment, the providing step includes providing an array of transducers. The manipulating step includes activating a first element of the array of transducers at a first time and a second of the array of transducers at a second time. The second time is delayed from the first time.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### BRIEF DESCRIPTION OF THE DRAWINGS

The accompanying drawings, which are incorporated in and form a part of the specification, illustrate the embodiments of the present invention and together with the written description serve to explain the principles, characteristics, and features of the invention. In the drawings:

FIG. 1 is a schematic illustrating an ultrasound transducer with a first embodiment of a sound block;

FIG. 2 is a schematic illustrating the ultrasound transducer of FIG. 1 with a second embodiment of a sound block with a convex lens;

FIG. 3 is a schematic illustrating the ultrasound transducer of FIG. 1 with a third embodiment of a sound block with a concave lens;

FIG. 4 is a schematic of an array transducer with annular elements; and

FIG. 5 is a schematic of a transducer with a mechanical time delay.

### DETAILED DESCRIPTION OF THE EMBODIMENTS

The following description of the preferred embodiment(s) is merely exemplary in nature and is in no way intended to limit the invention, its application, or uses.

The diffraction pattern of the ultrasound transducer has two characteristic zones: the near field (closer to the transducer) and the far field (farther from the transducer). The transition between these zones for a circular (piston) transducer is well known as the quotient between the transducer's active area radius and the ultrasound wavelength in the medium. The biological response is optimized in a range of distances in the far field, but the distance between the far field and the transducer depends on the geometry of the transducer, the frequency of the ultrasound wave and the medium through which the wave travels. The different embodiments presented here bring the transition between the far and near field closer to the transducer by varying several of these parameters except for the frequency of the wave.

The cellular response to ultrasound is generally understood. For example, experimental ST-2 pre-osteoblastic cell line was exposed to different parts of the ultrasound generated by the ultrasound transducer. The distances from the transducer were varied by placing an array of transducers in a tank of water and varying the distance of the tissue culture vessel holding the cells. The distances tested were 0 mm, 60 mm and 130 mm. The distance of 130 mm placed the cells into the ultrasound far-field.

The activation of cell signaling generally utilizes the phosphorylation of tyrosine residues on key proteins, such as FAK and Erk. These proteins have previously been shown to be phosphorylated upon exposure to low intensity pulsed ultrasound and implicated with the osteogenic response of cells to low intensity pulsed ultrasound stimulus.

The results from these experiments show that cells exposed to low intensity pulsed ultrasound in the far field, 130 mm away from the transducer, have greater amounts of phosphorylated tyrosine residues compared with those stimulated at 0 mm or 60 mm from the transducer. The greater amounts of phosphorylated tyrosine relates directly to the level of activated tyrosine residues on proteins within the cells and indicates the greater level of activation in cells stimulated within the far field of low intensity pulsed ultrasound.

Whether the far field of the low intensity pulsed ultrasound could be beneficial in fracture repair was tested. The theory was tested with the rat closed femoral fracture model developed by Einhorn (Boneres and Einhorn). This model is the only true representation in vivo of a traumatic fracture and is directly comparable to the traumatic fractures seen in humans. After the fracture was created, the animals were treated with low intensity pulsed ultrasound with either the transducer in contact with the skin or the fracture within the far field. Surprisingly, the bone fractures in animals treated with low intensity pulsed ultrasound in the far field showed a significantly greater strength when tested under torsion. This increase in strength of the bone fractures correlates to beneficial healing and acceleration of the fracture healing cascade.

A method or an apparatus to manipulate the location of the far field of the transducer is disclosed. Exemplary apparatus for manipulation of the far field are described below.

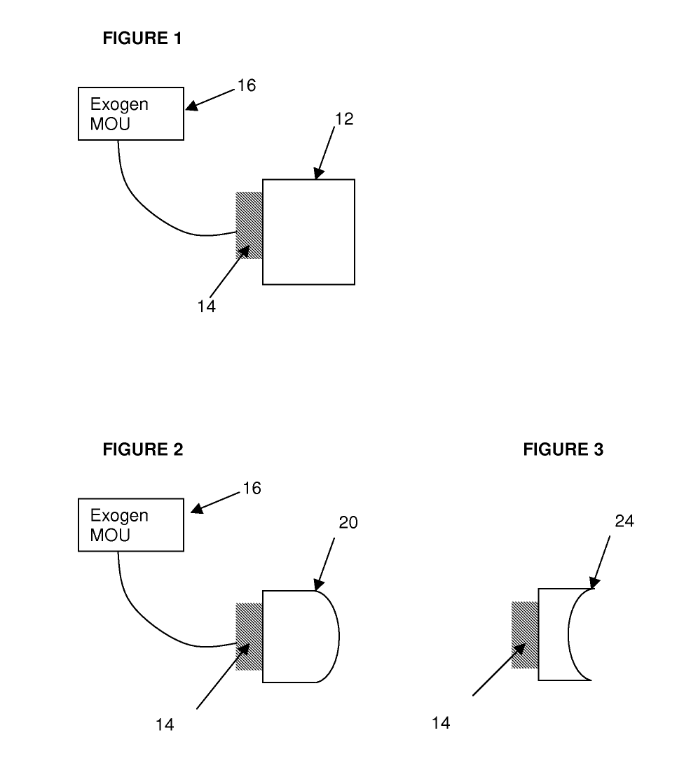

FIG. 1 is a schematic illustrating an ultrasound transducer 14 with a first embodiment of a sound block 12. The sound block 12 is placed on an ultrasound transducer 14. The transducer 14 is electrically connected to a control unit 16, for example an Exogen MOU controller. In FIG. 1, the sound block 12 is a far field applicator and includes a block of material with a very high speed of sound. The speed of sound of the block 12 may be less than, equal to, or greater than the speed of sound of the tissue for treatment. The block 12 changes the diffraction pattern, effectively shrinking the diffraction pattern in the near field and thus moving the transition between the far field and the near field closer to the transducer 14. The block 12 may preferably have similar acoustic impedance to that of water to minimize reflections at the interface between the block 12 and transducer 14. It may be made of a single material or made up of layers parallel to the transducer 14 surface with different speeds of sound or acoustic impedance. As an example only, the block 12 may be made from aluminum. The size of the block 12 and/or the material may be selected to treat a specific type of fracture. Moreover, the size of the block 12 and/or the material may be selected to place the far field at a certain distance from the face of the transducer 14.

The size of the block 12, both in depth from the transducer surface and width across the transducer surface, effects the near and far field properties. The depth of the block 12 effects the depth to the far field inversely; the deeper the block 12, the closer to the tissue surface the far field is achieved. The width of the block 12 effects the far field because the waves, as they travel to the edges of the block 12, are reflected back into the block and interfere with the waves traveling through the block 12. While this example discusses blocks 12 with perpendicular surfaces, as described below, curved surfaces may also be used to effect the transition between the near field and far field.

In FIGS. 2 and 3, the far field applicator consist of a block of material with a curved surface that changes the shape of the beam to move point of maximum acoustic intensity in the far field closer to the transducer. FIG. 2 is a schematic illustrating the ultrasound transducer 14 of FIG. 1 with a second embodiment of a sound block 20 with a convex lens. FIG. 3 is a schematic illustrating the ultrasound transducer of FIG. 1 with a third embodiment of a sound block with a concave lens 24. The blocks 20 and 24 effectively moves the far field and the near field closer to the transducer 14. The blocks 20 and 24 will preferably have similar acoustic impedance to that of water to minimize reflections at the interface between the block and the tissue. The outside surface of the blocks 20 and 24 can have a spherically convex or concave surface depending on the speed of sound and acoustic impedance of the material.

The concave and convex shapes of the blocks 20 and 24 change the time for a wave to be transmitted at the surface of the far field applicator. As the wave is transmitted from the transducer surface, the time it takes the wave to travel to a point at a given distance from the transducer surface is a combination of the time to travel through the far field applicator and the time to travel through the space between the surface of the far field applicator and the point of interest. The overall time to travel to the point is less for a path that travels through a longer portion of the far field applicator because the speed of sound through the far field applicator is greater than the speed of sound through the tissue under the transducer. These shapes for blocks may also be combined, for example, by changing the material of the block and then adjusting the lens, size, or depth of the blocks.

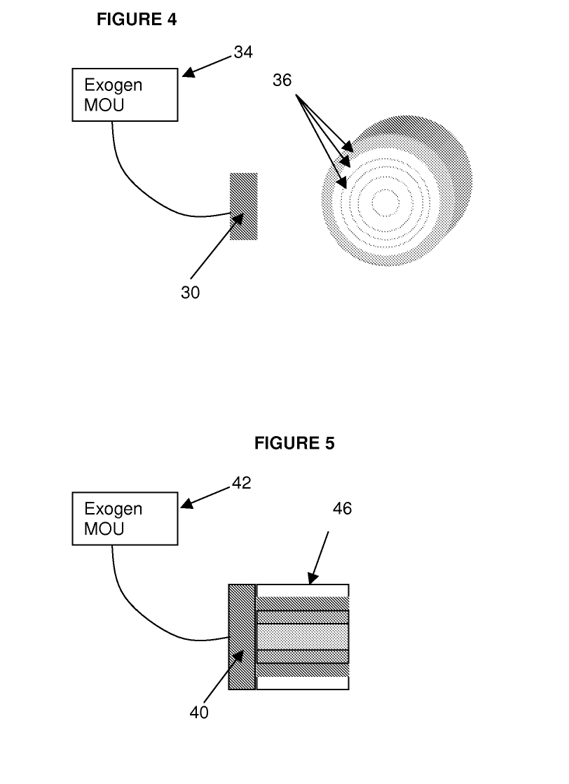

FIG. 4 is a schematic of an array transducer 30 with annular elements 32. The transducer 30 is implemented as the array of annular elements 32 may be activated individually or grouped together. A controller 34 may activate a subset of these elements 32 at the same time, effectively changing the active aperture and thus changing the distance of the far field to near field transition. This effect is achieved by activating groups of elements 32 that form a larger or smaller active circular radiating area. These array elements 32 can be implemented as rings of piezoelectric material separated by an isolation gap with separate connections or by having patterned electrodes (annular shape) on a piezoelectric disc. These elements could also be annular sectors implemented by Micro-Electro-Mechanical-Systems (MEMS) with separate or grouped connections. In effect, the time delay between the array elements acts as a lens to focus the energy to obtain the far field effect.

In alternative embodiment based on the embodiment of FIG. 4, each annular element 32 may be activated using a different voltage to manipulate the diffraction pattern using a well known technique called aperture apodization.

In an alternative embodiment based on the embodiment of FIG. 4, each annular element 32 can be activated using a different voltage and applying a different time delay to the drive signal to manipulate the diffraction pattern using a well known technique used for focusing the ultrasound beam in imaging systems.

In the embodiment depicted in FIG. 5, the far field applicator includes a cylindrical block made of concentric tubes made of materials with different speeds of sound to effectively achieve the same effect mechanically as described above. The outer surface of the applicator can be parallel to the transducer or be convex or concave depending on the speed of sound distribution of the mechanical array elements. The number and dimensions of the tube elements depends on the application for which this device is used. The speed of sound should gradually decrease or increase from the center material to the outer layer.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A system for ultrasound therapy, comprising:
a transducer configured to deliver an ultrasound wave to tissue, and
means for manipulating the diffraction pattern to alter the transition point between the near field and far field.

2. The system of claim 1, wherein the means for manipulating the diffraction pattern includes a block coupled to the transducer, the block having a speed of sound for propagating waves different from the speed of sound for propagating waves of the tissue.

3. The system of claim 2, wherein the speed of sound for propagating waves in the block is greater that the speed of sound of propagating waves in the tissue.

4. The system of claim 1, wherein the means for manipulating the diffraction pattern includes a lens.

5. The system of claim 4, wherein the lens is a concave lens.

6. The system of claim 1, wherein the means for manipulating the diffraction pattern includes an array of transducers.

7. The system of claims 6, wherein the array of transducers are concentric.

8. The system of claim 6, wherein the array of transducers are activated by varying the voltage to each element in the array of transducers.

9. The system of claim 6, wherein a first element in the array of transducers is activated after a time delay from the activation of a second element in the array of transducers.

10. The system of claim 1, wherein the means for manipulating the diffraction pattern includes a first block having a first speed of sound for propagating waves and a second block having a second speed of sound for propagating waves, the second speed of sound for propagating waves being different than the first speed of sound for propagating waves.

11. The system of claim 10, wherein the first block and second block are concentric.

12. A method for ultrasound treatment of tissue, comprising: providing a transducer for propagating ultrasound waves into tissue; identifying a depth for treatment; and manipulating the diffraction pattern of the ultrasound waves such that the transition between the near field and the far field is between the transducer and the identified depth of treatment.

13. The method of claim 12, wherein manipulating includes propagating the ultrasound signal through a lens.

14. The method of claim 12, wherein providing includes providing an array of transducers and manipulating includes activating a first element of the array of transducers with a first voltage and a second of the array of transducers with a second voltage, the second voltage being different from the first voltage.

15. The method of claim 12, wherein providing includes providing an array of transducers, and manipulating includes activating a first element of the array of transducers at a first time and a second of the array of transducers at a second time, the second time being delayed from the first time.
